(19) United States
(12) Patent Application Publication
(10) Pub. No.: US 2017/0135606 A1
(43) Pub. Date: May 18, 2017

(54) SYSTEM, DEVICE, AND METHOD FOR MEASURING NET LOAD ON A LOWER EXTREMITY

(71) Applicant: UNIVERSITY OF UTAH RESEARCH FOUNDATION, Salt Lake City, UT (US)

(72) Inventors: Robert W. Hitchcock, Sandy, UT (US); Tomasz J. Petelenz, Salt Lake City, UT (US); Kylee North, Bountiful, UT (US); Erik N. Kubiak, Salt Lake City, UT (US)

(21) Appl. No.: 15/355,331

(22) Filed: Nov. 18, 2016

Related U.S. Application Data
(60) Provisional application No. 62/257,114, filed on Nov. 18, 2015.

(51) Int. Cl. A61B 5/103; A61B 5/00; A43B 23/07; A43B 3/00; A43B 13/14; A43B 3/02

(52) U.S. Cl. CPC A61B 5/1038; A43B 13/141; A43B 3/02; A43B 23/07; A43B 3/0005; A61B 5/6807; A61B 5/0002; A61B 2090/064

(57) ABSTRACT

Implementations of the present disclosure relate to apparatuses, systems, and methods for measuring net load on a lower extremity. In one embodiment a bootcast may include a body and a sole. The sole may be configured to receive at least a first force. The body may be configured to receive at least a second force. One or more first sensors may be located in or on the sole and may be configured to measure the first force. One or more second sensors may be located in or on the body and may be configured to measure the second force.

### CROSS-REFERENCE TO RELATED APPLICATIONS

This application claims priority to U.S. Provisional Patent Application No. 62/257,114, filed Nov. 18, 2015, the entire contents of which is incorporated by reference herein.

### BACKGROUND OF THE DISCLOSURE

The most common long bone fracture in the lower limb is a fracture of the tibia. A tibia fracture is particularly difficult to treat and rehabilitate due to high rates of infection and/or delayed healing. Delayed healing of tibia fractures may result in significant socioeconomic impacts to the patient in terms of increased healthcare costs, lost work productivity, extended rehabilitative resources, and permanent disabilities. Rehabilitation of tibia fractures involves progressively increasing the amount of weight placed on the injured leg in a process known as “limb loading.”

Little to no objective data exists to guide the rehabilitation of lower extremity fractures, such as a tibia fracture. There exists no viable means by which to direct graduated limb loading and thereby guide this type of rehabilitation. Graduated limb loading uses incremental increases in net load on a lower extremity to encourage osteogenesis and proper healing of the bone without incurring additional injury to the limb. Net load is the amount of a patient's weight that is borne by the injured extremity. For example, a net load may be zero when a patient uses crutches. A walking cast may allow the patient to apply some force to the lower extremity, but the cast itself may bear a portion of the weight. The amount of the patient's weight borne by the limb is the net load. Limb loading rehabilitation may also include varying the amount of time a load is applied to the lower extremity in conjunction with the nominal amount of force applied at any given time.

Monitoring the net load on a lower extremity continuously may allow medical professionals to more precisely guide rehabilitation of the fracture. An accurate measurement of the net load on the lower extremity and the amount of force borne by a bootcast in both stationary and ambulatory conditions may be desirable. Collection of such information during extended periods of time during a patient's daily activity may also provide medical professionals with valuable information for providing more precise rehabilitation.

### BRIEF SUMMARY OF THE DISCLOSURE

This summary is provided to introduce a selection of concepts that are further described below in the detailed description. This summary is not intended to identify specific features of the claimed subject matter, nor is it intended to be used as an aid in limiting the scope of the claimed subject matter.

In one or more embodiments, a bootcast includes a body and a sole. The sole is configured to receive at least a first force and the body is configured to receive at least a second force. The bootcast includes one or more first sensors that are located in or on the sole and configured to measure the first force. The bootcast also includes one or more second sensors that are located in or one the body and configured to measure the second force.

In one or more embodiments, a bootcast includes a body and a sole. The sole is configured to receive at least a first force and the body is configured to receive at least a second force. The bootcast includes one or more first sensors that are located in or on the sole and configured to measure the first force. The bootcast also includes one or more second sensors that are located in or on the body and configured to measure the second force. The one or more first sensors and one or more second sensors are in data communication with a data storage device configured to store the data of the measured first force and second force.

In one or more embodiments, a method of calculating a net load on a lower extremity includes enclosing a lower extremity inside a bootcast having one or more force sensors therein. A first force is then applied to at least one force sensor and a second force is applied to at least one other force sensor. The method further includes measuring the first force using the at least one force sensor and measuring the second force using the at least one other force sensor to determine the net load on the lower extremity.

Additional features of embodiments of the disclosure will be set forth in the description which follows. The features of such embodiments may be realized by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1 is a side view of a bootcast including integrated force sensors, according to at least one embodiment described herein;

FIG. 2 is a side cross-sectional view of a bootcast including integrated force sensors in a body and sole thereof, according to at least one embodiment described herein;

FIG. 3 is a side view of a bootcast having one or more rigid support members and one or more resilient support members therein, according to at least one embodiment described herein;

FIG. 4 is a perspective view of a flexible sole having one or more force sensors therein, according to at least one embodiment described herein;

FIG. 5 is a side view of a flexible bootcast having one or more rigid support members, one or more resilient support members, and one or more force sensors therein, according to at least one embodiment described herein;

FIG. 6 is a side view of a bootcast having one or more force sensors in data communication with a data storage device and in electrical communication with an energy storage device, according to at least one embodiment described herein; and

FIG. 7 is a flowchart depicting a method of calculating a net load on a lower extremity.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to constructing and using a bootcast having integrated force sensors to measure the force applied to a plurality of locations on the bootcast. The bootcast may have a body (i.e., an upper portion) and a base (i.e., a lower portion). The base may have one or more sensors in a sole that may measure a first force applied by a patient's foot to the sole and transmitted through the sole to the ground. The body may have one or more sensors that may measure a second force applied by the patient's leg to the bootcast and transmitted by the bootcast to the ground. The first force may be the net load on the patient's lower extremity. The second force may provide information regarding the total force applied to the patient's leg during rehabilitation. The bootcast may be a rigid bootcast or may be a flexible bootcast with structural members that transmit the second force from the upper portion of the bootcast to the ground. The bootcast may include one or more energy storage devices and/or one or more data storage devices to collect and store information regarding forces applied to the bootcast by the patient's lower extremity over time.

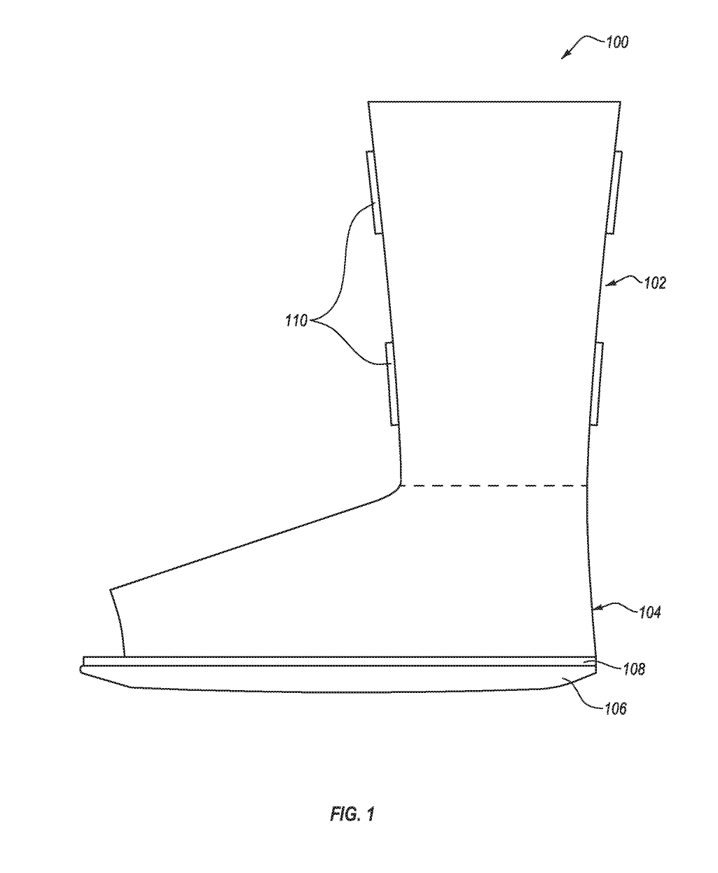

FIG. 1 is a side view of a bootcast 100 including integrated force sensors, according to at least one embodiment as described herein. The bootcast 100 may have a body 102 and a base 104. The body 102 and base 104 may, in some embodiments, be modular, allowing different sizes of bodies 102 and bases 104 to be interchanged. A modular bootcast 100 may accommodate a broader range of patient morphologies and allow the bootcast 100 to be reusable between multiple patients. The base 104 of the bootcast 100 may have a tread 106 and a sole 108. The tread 106 may be a rubber, elastomer, synthetic, leather, other material that may provide adequate traction with the ground, or combinations thereof. The sole 108 may include one or more force sensors that may measure a first force from a patient's foot.

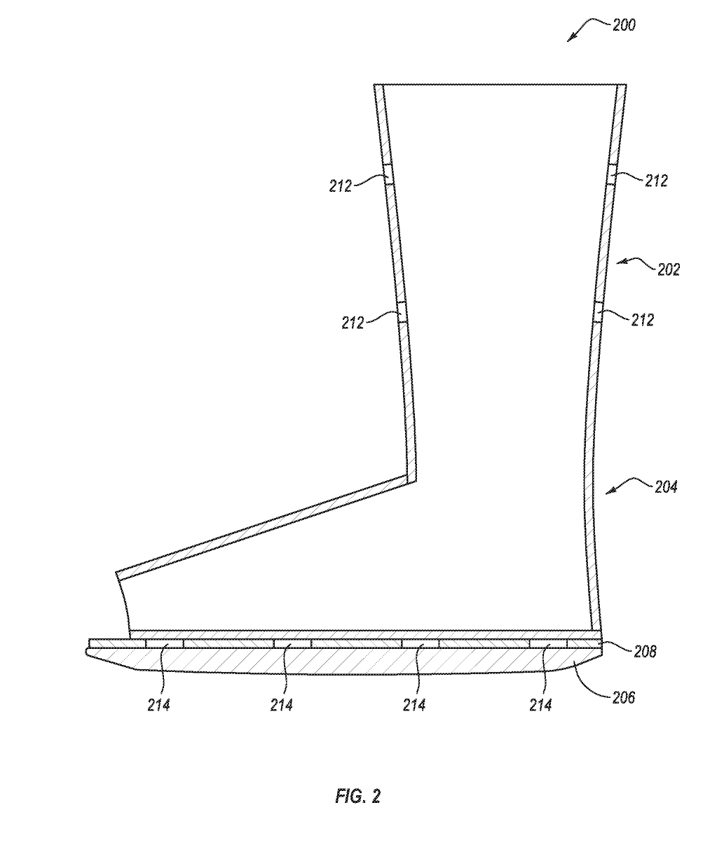

The body 102 of the bootcast 100 may include a plurality of body sensors 110 connected to or incorporated into the body 102. In some embodiments, body sensors 110 may be force sensors that measure force normal to a surface of the body 102 and/or may be force sensors that measure force transmitted in line with the wall or walls of the body 102 to the ground (such as integrated body sensors 212 described in more detail in relation to FIG. 2). For example, the body sensors 110 may be connected to the front and rear of the body 102 and may be substantially aligned with the patient's shin and calf. Substantially aligned may include being parallel with the patient's shin and calf. Substantially aligned may include being between 0° and 20° from parallel. For example, substantially aligned may include being between 0° and 15°, between 0° and 10°, and 0° and 5° from parallel.

The body sensors 110 may be positioned on the body 102 such that the body sensors 110 at least partially oppose one another. In the depicted embodiment, the body sensors 110 are oriented approximately 180° from one another. In other embodiments, one or more body sensors 110 may be oriented at least about 90° from at least one other body sensor 110. For example, each of the plurality of body sensors 110 may measure a force vector applied to the body sensor 110. The plurality of body sensors 110 may be configured such that each body sensor 110 measures a force vector at least 90° from another force vector measured by another body sensor 110. The body sensors 110 may measure vertical force (e.g., gravitational force) applied to the body 102 by a patient's leg and/or measure other forces (e.g., lateral forces) applied to the body 102 by the patient's leg while stationary and/or during walking or other ambulatory movements. In other embodiments, the simultaneous measurement of forces at different positions on the body 102 may allow for the measurement of a torque applied to the patient's leg. In yet other embodiments, the plurality of body sensors 110 may include accelerometers that may measure acceleration (e.g., movement and/or impacts) of the patient's leg and/or bootcast 200

FIG. 2 is a side cross-sectional view of a bootcast 200 having integrated force sensors, according to at least one embodiment as described herein. The bootcast 200 may have a body 202 and a base 204. The body 202 and/or the base 204 may be adjustable. For example, the body 202 may be adjustable to apply differing amounts of pressure on the patient's leg and/or adjust to different sizes of patients' legs. The base 204 may be adjustable to apply differing amounts of pressure on the patient's foot and/or adjust to different sizes of patients' feet. In another example, the body 202 and/or base 204 may be adjustable to accommodate a patient wearing footwear, such as a shoe, a sock, another insulating layer, or other protective footwear inside the bootcast 200. Adjusting the body 202 tighter against the patient's leg may restrict the downward movement of the patient's leg toward the base 204.

The body 202 may include one or more body sensors 212. In some embodiments, the one or more integrated body sensors 212 may be similar to the one or more body sensors 110 described in relation to FIG. 1, or, in other embodiments such as that shown in FIG. 2, the one or more integrated body sensors 212 may be integrated into the body 202 to measure the force applied downward on the body 202 and transmitted by the bootcast 200 to the ground. The one or more integrated body sensors 212 may measure a portion of the total load from a patient on the bootcast 200 that is borne by the body 202 and does not contribute to a net load on the lower extremity. The portion of the total load from the patient on the bootcast 200 that is borne by the body 202 may be transmitted to the ground through a tread 206 and may bypass a sole 208. For example, adjusting the body 202 tighter against the patient's leg may reduce a first force measured by one or more sole sensors 214. The sole 208 may therefore receive the remaining portion of the total load from the patient on the bootcast 200 that is not borne by the body 202 and is transmitted to the sole 208 through the patient's lower leg and foot.

The bootcast 200 may have a sole 208 that is at least partially located between the patient's foot and the ground. In some embodiments, the sole 208 may be integrated into the base 204. In other embodiments, the sole 208 may be separate from the base 204 and configured to be positioned between the base 204 and the tread 206. In yet other embodiments, the sole 208 may be a separate component from the base 204 and may be configured to fit inside at least part of the base 204. The sole 208 may include a plurality of sole sensors 214. The sole sensors 214 may be force sensors that may measure the first force applied by the patient's foot to the ground through the sole 208. The sole sensors 214 may also include accelerometers to measure acceleration (e.g., movement and/or impacts) of the foot and/or bootcast 200.

In some embodiments, the body 202 and/or base 204 may be rigid. The body 202 and/or base 204 may resist deformation and/or deflection in a variety of degrees of freedom. For example, the body 202 and/or base 204 may resist deformation and/or deflection in a first direction in line with a walking stride (i.e., forward and backward from a patient's perspective). In another example, the body 202 and/or base 204 may resist deformation and/or deflection in a second direction transverse to the first direction (i.e., a lateral direction from a patient's perspective). In yet another example, the body 202 and/or base 204 may resist deformation and/or deflection about a rotational axis (i.e., a torque applied about the leg from a patient's perspective). In a yet further example, the body 202 and/or base 204 may resist deformation and/or deflection in a third direction vertical to the bootcast 200 (i.e., a compressive force downward from a patient's perspective). The body 202 and/or base 204 may resist deformation and/or deflection in any combination of the described first, second, third, or rotational directions.

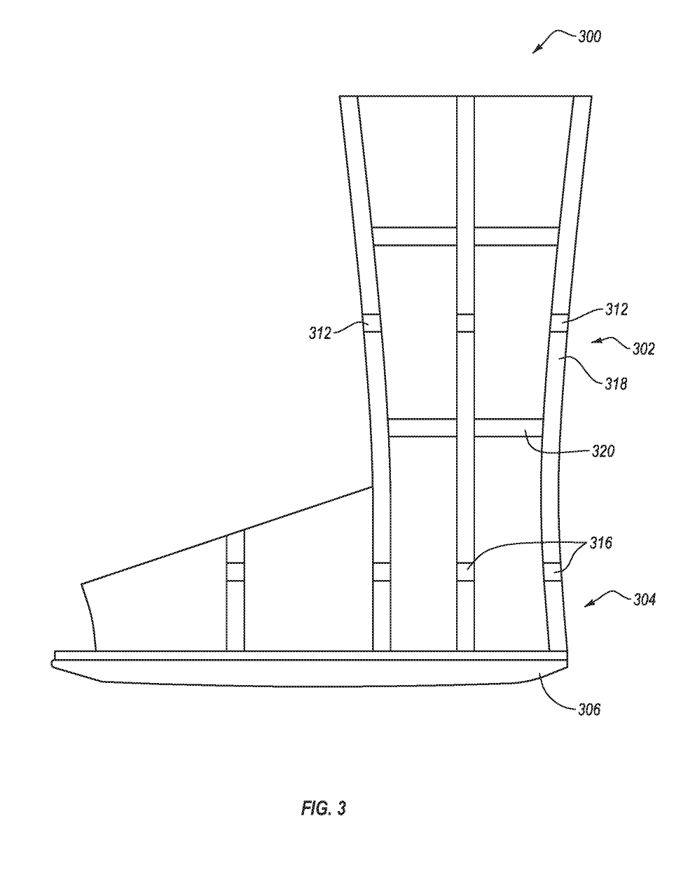

FIG. 3 depicts a bootcast 300 having a body 302 and a base 304 that may be flexible in one or more directions and rigid in one or more directions to allow different degrees of movement for a patient. The bootcast 300 may include one or more integrated body sensors 312 in the body 302 and one or more integrated base sensors 316 in the base 304. In some embodiments, the body 302 and/or base 304 may include one or more rigid support members 318. In other embodiments, the body 302 and/or base 304 may include one or more resilient support members 320. In the depicted embodiment, the body 302 and base 304 include one or more rigid support members 318 and one or more resilient support members 320. The one or more rigid support members 318 may be substantially rigid and configured to transmit force therethrough. For example, the one or more rigid support members 318 depicted in FIG. 3 may be configured to transmit force applied to the body 302 of the bootcast 300 downward to the tread 306 and/or ground. The one or more rigid support members 318 may, thereby, provide support to the patient's lower extremity and reduce a net load on the injured area of the patient's leg. The one or more resilient support members 320 may be at least somewhat flexible and may deform when a force is applied thereto. For example, the one or more resilient support members 320 may provide flexible support to the one or more rigid support members 318, allowing the bootcast 300 to be flexible and/or compressible in at least one direction (i.e., a radial direction) while the bootcast 300 may be substantially rigid in another direction (i.e., a vertical direction).

While the depicted embodiment of a bootcast 300 illustrates the one or more rigid support members 318 in a vertical arrangement, it should be understood that, in other embodiments, a bootcast 300 may include one or more rigid support members 318 in other orientations. For example, the one or more rigid support members 318 may be oriented in a vertical orientation, a horizontal orientation, any orientation therebetween, or combinations thereof. One or more rigid support members 318 may form a junction at one or more locations on the bootcast 300. In some embodiments, one or more rigid support members 318 may form a junction having an angular relationship of 10°, 20°, 30°, 40°, 50°, 60°, 70°, 90°, or any value therebetween. For example, the one or more rigid support members 318 may form a junction of between 10° and 90°. In another example, the one or more rigid support members 318 may form a junction of between 20° and 60°. In yet another example, the one or more rigid support members 318 may form a junction of between 30° and 50°.

While the depicted embodiment of a bootcast 300 illustrates the one or more resilient support members 320 in a horizontal arrangement, it should be understood that, in other embodiments, a bootcast 300 may include one or more resilient support members 320 in other orientations. For example, the one or more resilient support members 320 may be oriented in a vertical orientation, a horizontal orientation, any orientation therebetween, or combinations thereof. One or more resilient support members 320 may form a junction at one or more locations on the bootcast 300. In some embodiments, one or more resilient support members 320 may form a junction having an angular relationship of 10°, 20°, 30°, 40°, 50°, 60°, 70°, 90°, or any value therebetween. For example, the one or more resilient support members 320 may form a junction of between 10° and 90°. In another example, the one or more resilient support members 320 may form a junction of between 20° and 60°. In yet another example, the one or more resilient support members 320 may form a junction of between 30° and 50°.

While the depicted embodiment of a bootcast 300 illustrates the one or more rigid support members 318 oriented at approximately 90° to the one or more resilient support members 320, it should be understood that, in other embodiments, a bootcast 300 may include one or more rigid support members 318 oriented at other angles to the one or more resilient support members 320. For example, one or more rigid support members 318 may be oriented relative to one or more resilient support members 320 to form a junction having an angular relationship of 10°, 20°, 30°, 40°, 50°, 60°, 70°, 90°, or any value therebetween. For example, one or more rigid support members 318 and one or more resilient support members 320 may form a junction of between 10° and 90°. In another example, one or more rigid support members 318 and one or more resilient support members 320 may form a junction of between 20° and 60°. In yet another example, one or more rigid support members 318 and one or more resilient support members 320 may form a junction of between 30° and 50°.

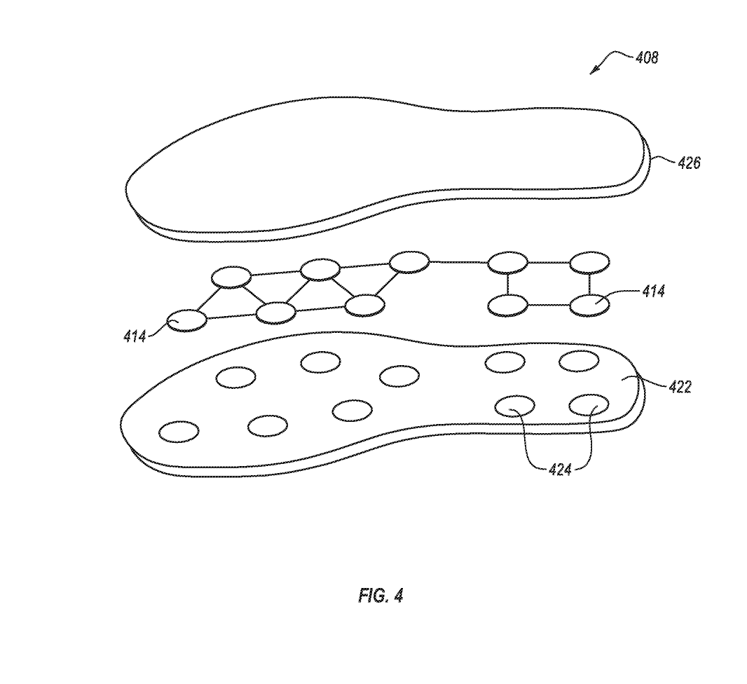

A bootcast may include one or more flexible materials that may allow at least a portion of a sole to flex. FIG. 4 depicts a flexible sole 408 that may be used in a bootcast, according to at least one embodiment described herein. The flexible sole 408 may have one or more sole sensors 414 incorporated into the sole 408 or positioned on the sole 408. In some embodiments, the sole 408 may have a lower sole 422 with one or more apertures 424 (e.g., openings, pockets, recesses, etc.) therein. The one or more apertures 424 may be configured to at least partially receive one or more sole sensors 414. In other embodiments, one or more apertures 424 may be positioned in an upper sole 426. In yet other embodiments, the one or more apertures 424 may be positioned in the lower sole 422 and the upper sole 426. For example, the lower sole 422 and the upper sole 426 may have at least one aperture 424 in each that is configured to partially receive a sole sensor 414 such that the apertures 424 in the lower sole 422 and the upper sole 426 cooperate to provide a single aperture 424 configured to receive and/or house a sole sensor 414.

In some embodiments, at least one of the one or more sole sensors 414 may be positioned on a surface of a lower sole 422 and/or upper sole 426 independently of an aperture 424. For example, a sole sensor 414 may be affixed to a surface of an upper sole 426 having no apertures 424 therein. The sole sensor 414 may, therefore, receive a direct application of force from the patient's foot with less distribution of force across other portions of the sole 408.

In some embodiments, at least one sole sensor 414 may be in data and/or electrical communication with at least one other sole sensor 414. In other embodiments, at least one sole sensor 414 may be in data and/or electrical communication with a plurality of other sole sensors 414. In yet other embodiments, at all of the sole sensors 414 in data and/or electrical communication with all of the other sole sensors 414. The sole sensors 414 may communicate through a wired connection or a wireless communication protocol. The sole sensors 414 may communicate information between the sole sensors 414 to provide a measurement of net load applied to the sole 408 by the patient's during different positions and/or movements (e.g., walking forward, walking laterally, standing, walking on uneven surfaces, etc.).

The lower sole 422 and upper sole 426 may be fixed relative to one another. In some embodiments, the lower sole 422 may be fixed to the upper sole 426 about a periphery of the lower sole 422 and the upper sole 426. In other embodiments, the lower sole 422 may be fixed to the upper sole 426 at selected locations (i.e., array of bonds) across a surface of the lower sole 422 and upper sole 426 to allow movement of some portions of the lower sole 422 and upper sole 426 relative to one another during flexion of the sole 408. In yet other embodiments, the lower sole 422 may be fixed to the upper sole 426 at substantially the entire surface of the lower sole 422 in contact with the upper sole 426. The lower sole 422 and upper sole 426 may be fixed relative to one another by any appropriate connection including, but not limited to, friction bonding, ultrasonic bonding, heat bonding, chemical bonding, adhesives, mechanical fasteners (e.g., stiches, staples, rivets, pins, etc.), or combinations thereof.

In some embodiments, the one or more sole sensors 414 may be fixed to the lower sole 422 and/or upper sole 426. For example, the one or more sole sensors 414 may be fixed to the lower sole 422 and/or upper sole 426 by any appropriate connection including, but not limited to, friction bonding, ultrasonic bonding, heat bonding, chemical bonding, adhesives, mechanical fasteners (e.g., stiches, staples, rivets, pins, etc.), or combinations thereof. In other embodiments, the one or more sole sensors 414 may be substantially retained by or within one or more apertures 424, but not fixed therein.

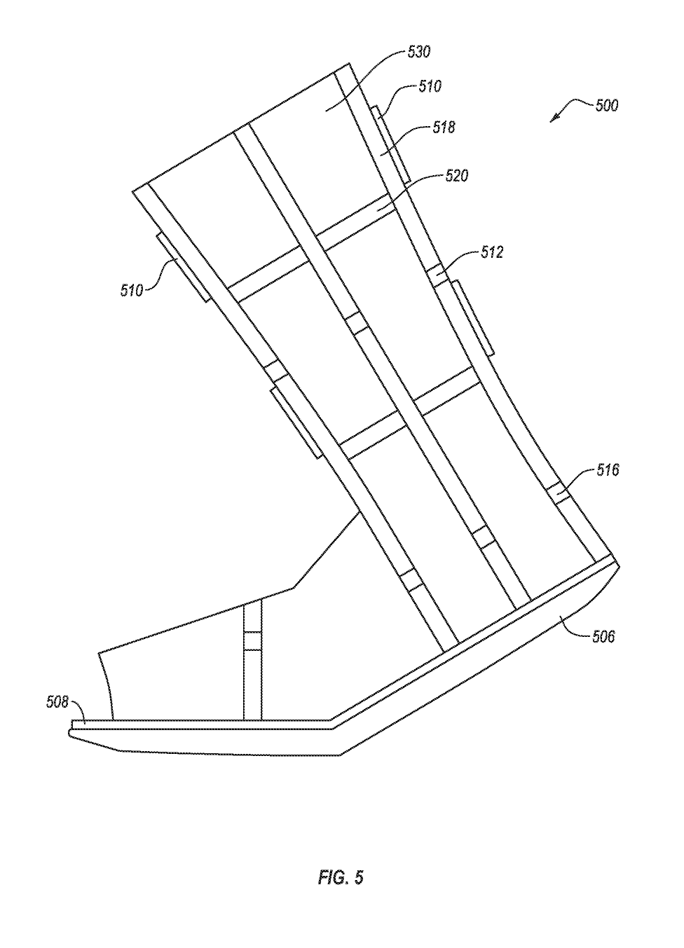

FIG. 5 depicts another embodiment of a bootcast 500 according to the present disclosure. A bootcast 500 may have a flexible inner liner 530 that may partially or entirely surround the lower extremity of a patient. For example, the inner liner 530 may surround the foot and lower leg of the patient. In another example, and as shown in FIG. 5, the inner liner 530 may have an open toe. In yet another example, the inner liner 530 may have one or more openings therethrough to reduce weight and/or provide circulation of air through the bootcast 500 for ventilation and/or comfort. The inner liner 530 may apply and/or distribute force to the patient's lower extremity from one or more parts of the bootcast 500, such as one or more rigid support members 518 and/or one or more resilient support members 520. The one or more rigid support members 518 may transmit force with little to no deformation due to an applied force from the patient's lower extremity, as described herein. The one or more resilient support members 520 may provide support to the one or more rigid support members 518 and/or inner liner 530 while deforming at least partially under an applied force from the patient's lower extremity. For example, one or more resilient support members 520 and/or the inner liner 530 may deform during a walking motion or other flexion of the bootcast 500 by the patient. The one or more rigid support members 518 may remain in their original shape without substantial deformation and may transmit force through the one or more rigid support members 518. The transmission of force through the one or more rigid support members 518 may allow the bootcast 500 to alter, control, or otherwise manage a net load on the patient's lower extremity.

The bootcast 500 may include a plurality of sensors (e.g., force sensors, accelerometers, etc.) within the bootcast 500 to measure the application and/or transmission of force through the bootcast 500 and/or patient's lower extremity during movement. The bootcast 500 may include one or more body sensors 510, one or more integrated body sensors 512, one or more sole sensors 514, one or more integrated base sensors 516, or combinations thereof. In some embodiments, the one or more body sensors may be attached to or at least partially embedded within the inner liner 530 and configured to measure a force applied to a part of the inner liner by the patient's lower extremity.

The array of one or more body sensors 510, one or more integrated body sensors 512, one or more sole sensors 514, one or more integrated base sensors 516, or combinations thereof may allow the continuous monitoring of multiple conditions during therapy and/or daily activities for a patient. For example, the array of sensors may collect a net load transmitted to the one or more sole sensors 514, a supported load transmitted through the one or more rigid support members 518 to a tread 506 measured by the one or more integrated body sensors 512 and/or integrated base sensors 516, force applied to by the patient's leg against the bootcast 500 in a forward and/or rearward direction monitored by the one or more body sensors 510, or combinations thereof. The array of sensors may include one or more accelerometers to collect data simultaneously with the force sensors to correlate relative forces applied to portions of the bootcast 500 with accelerations (i.e., movement) of the bootcast 500. Information regarding relative changes in net load, supported load, torque, other measured forces, or combinations thereof during different activities or exercises may help medical professionals guide the therapy and healing of a lower extremity injury.

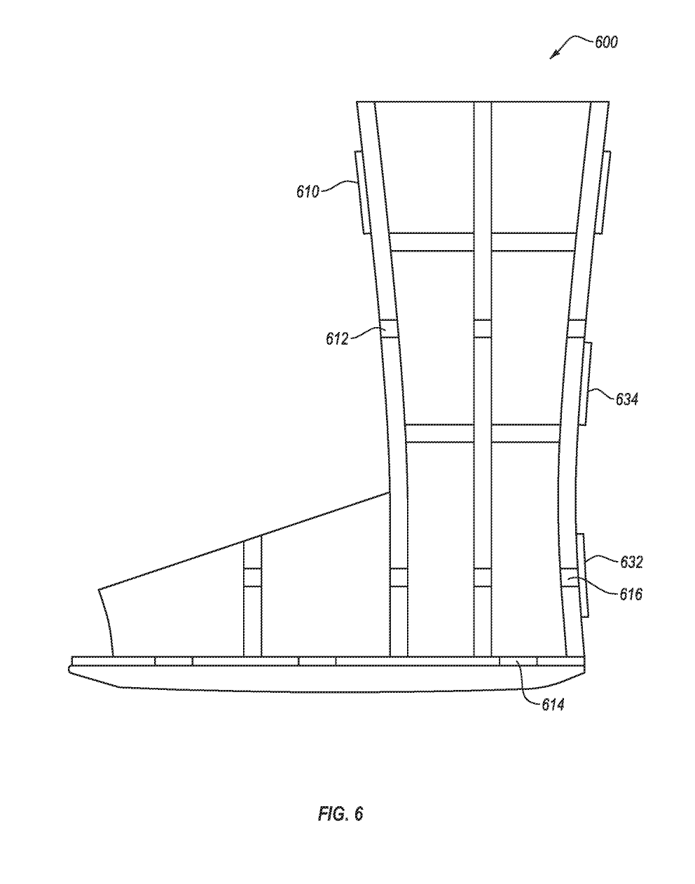

As shown in FIG. 6, a bootcast 600, according to at least one embodiment as described herein, may be configured to provide larger datasets to the medical professionals, and hence, more information to guide the therapy. The bootcast 600 includes an on-board energy storage device 632 and/or a data storage device 634. The energy storage device 632 may include any suitable structure for storing electrical energy, such as primary cell batteries; secondary cell batteries such as lead-acid batteries, lithium-ion batteries, nickel-cadmium batteries, nickel metal hydride batteries, flow batteries, polymer-based batteries, sodium-ion batteries, silver-zinc batteries, or fuel cells; capacitors; or combinations thereof. In some embodiments, the energy storage device 632 may be in electrical communication with and provide electrical energy to at least one of the one or more body sensors 610, one or more integrated body sensors 612, one or more sole sensors 614, one or more integrated base sensors 616, or combinations thereof. In other embodiments, the energy storage device 632 may be in electrical communication with and provide electrical energy to the data storage device 634.

The data storage device 634 may include a data storage medium including, but not limited to, semiconductor, magnetic, optical, phase-change material, molecular, holographic, other types of memory, or combinations thereof. In some embodiments, the data storage device 634 may include removable media and/or a data transmission device to transfer data from the data storage device 634 to a computing device for viewing and/or analysis of the collected data. The data storage device 634 may include one or more microprocessors to direct and control the transmission of data from the one or more body sensors 610, one or more integrated body sensors 612, one or more sole sensors 614, one or more integrated base sensors 616, or combinations thereof to the data storage device 634.

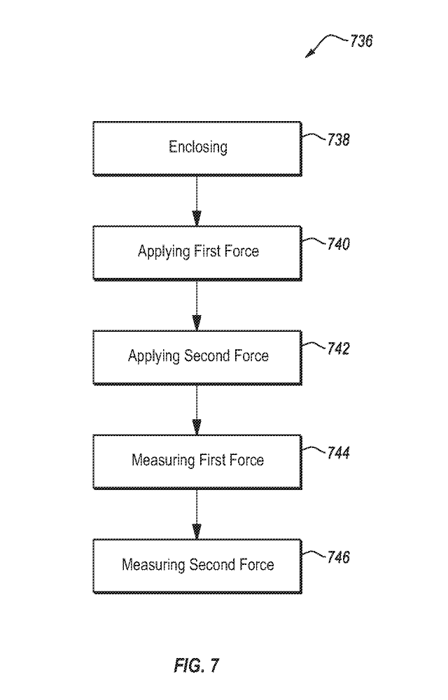

FIG. 7 is a flowchart depicting a method 736 for calculating a net load on a lower extremity. The method 736 includes enclosing 738 a lower extremity inside a bootcast having one or more force sensors therein and applying 740 a first force to at least one force sensor and applying 742 a second force to at least one other force sensor. The method 736 further includes measuring 744 the first force using the at least one force sensor and measuring 746 the second force using the at least one other force sensor. In some embodiments, the first force may be applied to one or more sole sensors in a sole of the bootcast and the second force may be applied to one or more body sensors and/or integrated body sensors in a body of the bootcast. In other embodiments, the method 736 may further include comparing the measured first force and the measured second force to one another. For example, comparing the measured first force and the measured second force may be done in a microprocessor built into the bootcast and in data communication with the at least one force sensor and the at least one other force sensor. In another example, comparing the measured first force and the measured second force to one another may be done by a remote computing device after the measured first force and the measured second force are stored in a data storage device in the bootcast. In some embodiments, the data stored in the data storage device may be transferred to a remote computing device by a physical connection (i.e., a wired connection) between the data storage device and the remote computing device, a removable storage media in the data storage device that may be physically transferred to the remote computing device, a wireless communication device that allows the stored data to be transmitted to the remote computing device, or combinations thereof.

In some embodiments, the method 736 may also include calibrating one or more force sensors in the bootcast. For example, the bootcast may apply a force or forces to the patient's lower extremity after the patient's lower extremity is enclosed by the bootcast. The bootcast may include one or more fastener mechanisms that allow for the selective securement or tensioning of the bootcast around the patient's lower extremity. The one of more force sensors in the bootcast may be calibrated (e.g., tared to zero) to account for and/or compensate for the force or forces applied by the bootcast to the patient's lower extremity.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A device comprising:
a bootcast having body and a sole, the sole being configured to receive at least a first force, the body being configured to receive at least a second force;
one or more first sensors located in or on the sole and configured to measure the first force; and
one or more second sensors located in or on the body and configured to measure the second force.

2. The device of claim 1, wherein the sole is a flexible sole.

3. The device of claim 1, wherein the sole further comprises an upper sole and a lower sole.

4. The device of claim 3, wherein at least one of the first sensors is located between the upper sole and the lower sole.

5. The device of claim 3, wherein at least one of the first sensors is located at least partially in the upper sole.

6. The device of claim 3, wherein at least one of the first sensors is located at least partially in the lower sole.

7. The device of claim 1, further comprising a data storage device in data communication with at least one of the first sensors and at least one of the second sensors.

8. A device comprising:
a boot having a body and a sole, the sole being configured to receive at least a first force, the body being configured to receive at least part of a user's leg and to receive at least a second force from the user's leg, wherein the body is configured to support at least a portion of a user's weight;
one or more first sensors located in the sole and configured to measure the first force;
one or more second sensors located in or on the body and configured to measure the second force; and
a data storage device in data communication with at least one of the first sensors and at least one of the second sensors.

9. The device of claim 8, wherein the body includes an inner liner configured to apply a compressive force to the user's leg.

10. The device of claim 9, wherein at least one of the second sensors is at least partially embedded within the inner liner.

11. The device of claim 8, wherein the sole further comprises an upper sole and a lower sole, at least one of the first sensors being embedded at least partially within the upper sole.

12. The device of claim 8, wherein the body comprises a plurality of second sensors, at least two of the plurality of second sensors substantially opposing one another.

13. A method comprising:
enclosing at least part of a user's leg in a boot having a body and a sole;
applying a first force to the sole;
applying a second force to the body;
measuring the first force using one or more first sensors located in the sole; and
measuring a second force using one or more second sensors located in the body.

14. The method of claim 13, further comprising comparing the first force to the second force.

15. The method of claim 13, wherein applying a first force comprises applying a first portion of a user's weight.

16. The method of claim 13, wherein applying a second force comprises applying a second portion of a user's weight.

17. The method of claim 13, further comprising storing the first force and the second force in a data storage device.

18. The method of claim 17, further comprising communicating data from the data storage device to a remote computer device.

19. The method of claim 13, wherein the body comprises a plurality of second sensors, at least two of the plurality of the second sensors substantially opposing one another and measuring the first force further comprises measuring a total force applied to the user's leg by the body.

20. The method of claim 13, further comprising calibrating the one or more second sensors after enclosing the at least a part of the user's leg.
